(19) United States
(12) Patent Application Publication
(10) Pub. No.: US 2008/0287319 A1
(43) Pub. Date: Nov. 20, 2008

(54) Separation Method and Apparatus of Single-Stranded Nucleic Acid, Microarray and Dna Chip

(75) Inventors: Tadashi Matsunaga, Tokyo (JP); Haruko Takeyama, Tokyo (JP); Tsuyoshi Tanaka, Tokyo (JP); Takeo Tanaami, Tokyo (JP); Saya Sato, Tokyo (JP); Hisao Katakura, Tokyo (JP); Yuji Mitsumori, Tokyo (JP); Takeyuki Mogi, Tokyo (JP)

Correspondence Address: SUGHRUE-265550, 2100 PENNSYLVANIA AVE. NW, WASHINGTON, DC, 20037-3213, US

(73) Assignee: YOKOGAWA ELECTRIC CORPORATION, Musashino-shi (JP)

(21) Appl. No.: 11/816,575

(22) PCT Filed: Feb. 27, 2006
(86) PCT No.: PCT/JP2006/303592

§ 371 Date: Aug. 17, 2007

(30) Foreign Application Priority Data
Feb. 28, 2005 (JP) 2005-052896

(51) Int. Cl. C40B 40/08; C07H 1/00

(52) U.S. Cl. 506/17; 536/25.4

(57) ABSTRACT

A separation method of single-stranded nucleic acid characterized in that nucleic acid amplification is performed using a first primer to which a second substance capable of binding specifically to a first substance is bound and a second primer to which the second substance is not bound, and double-stranded nucleic acid obtained by the nucleic acid amplification is bound to the first substance, and the double-stranded nucleic acid bound to the first substance is dissociated into a single strand, and a separation apparatus of single-stranded nucleic acid characterized by having a nucleic acid amplification part 1 for performing nucleic acid amplification using a first primer to which a second substance capable of binding specifically to a first substance is bound and a second primer to which the second substance is not bound, a binding part 2 for binding double-stranded nucleic acid obtained by the nucleic acid amplification to the first substance, and a dissociation part 3 for dissociating the double-stranded nucleic acid bound to the first substance into a single strand.

### TECHNICAL FIELD

The present invention relates to a separation method and apparatus of single-stranded nucleic acid, and a DNA chip and a microarray using single-stranded nucleic acid obtained.

### BACKGROUND ART

A DNA chip or a DNA microarray, etc. for hybridization are used in the case of measuring gene sequences of a biopolymer such as DNA, RNA or protein added to DNA (hereinafter description is made by taking DNA as an example).

In the case of analyzing gene information by hybridization using such a DNA microarray or a DNA chip, etc., it is necessary to prepare a target made of single-stranded nucleic acid with the amount necessary and sufficient for analysis from a test sample.

In preparation of such a target, for example, as described in Non-patent Reference 1, biotin-labeled cRNA is used as a target in CodeLink Bioarray of Amershambiosciences Corporation, but in preparation of this biotin-labeled cRNA, centrifugation treatment etc. are required plural times and the treatment is troublesome and also, necessary time becomes a relatively long time (1.5 days) and the overall cost is increased.

Non-patent Reference 1: Amershambiosciences Corporation, “High-performance Single Dye Microarray: CodeLink Bioarray”, [online], [Search on Jan. 14, 2005], Internet <URL:http://www.jp.amershambiosciences.com/technologies/mi croarrays/pdf/codelink.pdf>

### DISCLOSURE OF THE INVENTION

### Problems to be Solved by the Invention

Therefore, an object of the invention is to solve the problems described above, and is to provide a technique capable of providing single-stranded nucleic acid used in the case of analyzing gene information simply in a short time.

### Means for Solving the Problems

According to the invention, there is provided with a separation method of single-stranded nucleic acid, including the steps of:

performing nucleic acid amplification using a first primer to which a second substance capable of binding specifically to a first substance is bound and a second primer to which the second substance is not bound, and

binding double-stranded nucleic acid obtained by the nucleic acid amplification to the first substance, and

dissociating the double-stranded nucleic acid bound to the first substance into a single strand.

According to the invention, the double-stranded nucleic acid is dissociated into a single strand by alkali treatment.

According to the invention, the first substance is avidin, and the second substance is biotin.

According to the invention, the first substance is an antigen, and the second substance is an antibody.

According to the invention, the first substance is gold, and the second substance is thiol.

According to the invention, the first substance is a substance having an amino group, and the second substance is a substance having a group binding covalently to an amino group.

According to the invention, a carrier is bound to the first substance.

According to the invention, the carrier is one of a magnetic particle, a bead, a basal plate and a fiber.

According to the invention, the second primer has a labeled substance.

According to the invention, the labeled substance is a fluorescent substance.

According to the invention, nucleotide used in the nucleic acid amplification has a labeled substance.

According to the invention, the labeled substance is a fluorescent substance.

Further, according to the invention, there is provided with a separation apparatus of single-stranded nucleic acid including:

a nucleic acid amplification part for performing nucleic acid amplification with using a first primer to which a second substance capable of binding specifically to a first substance is bound and a second primer to which the second substance is not bound,

a binding part for binding double-stranded nucleic acid obtained by the nucleic acid amplification to the first substance, and

a dissociation part for dissociating the double-stranded nucleic acid bound to the first substance into a single strand.

Further, according to the invention, there is provided with a microarray having single-stranded nucleic acid obtained by the separation method of single-stranded nucleic acid as a probe.

Further, according to the invention, there is provided with a microarray wherein single-stranded nucleic acid obtained by the separation method of single-stranded nucleic acid is hybridized as a target.

Further, according to the invention, there is provided with a DNA chip wherein single-stranded nucleic acid obtained by the separation method of single-stranded nucleic acid is hybridized as a target.

### EFFECT OF THE INVENTION

The separation method and apparatus of single-stranded nucleic acid of the invention can provide single-stranded nucleic acid used in the case of analyzing gene information simply in a short time at low cost.

### BRIEF DESCRIPTION OF THE DRAWINGS

FIG. 1 is a flowchart of a separation method of single-stranded nucleic acid of the invention.

FIG. 2 is a diagram showing an outline of one example of a separation apparatus of single-stranded nucleic acid of the invention.

FIG. 3 is a diagram showing an outline of one embodiment of the separation method of single-stranded nucleic acid of the invention.

FIG. 4 is a diagram showing an outline of another embodiment of the separation method of single-stranded nucleic acid of the invention.

FIG. 5 is a diagram showing a result of experiment data in the present description.

### DESCRIPTION OF REFERENCE NUMERALS AND SIGNS

1 Nucleic Acid Amplification Part
2 BINDING PART
3 DISSOCIATION PART
4 MAGNETIC PARTICLE
5 MAGNET
11 FIRST PRIMER
12 SECOND PRIMER
13 dNTP
21 BIOTIN
22 FLUORESCENT SUBSTANCE
23 AVIDIN

### BEST MODE FOR CARRYING OUT THE INVENTION

A separation method of single-stranded nucleic acid of the invention is characterized in that nucleic acid amplification is performed using a first primer to which a second substance capable of binding specifically to a first substance is bound and a second primer to which the second substance is not bound, and double-stranded nucleic acid obtained by the nucleic acid amplification is bound to the first substance, and the double-stranded nucleic acid bound to the first substance is dissociated into a single strand.

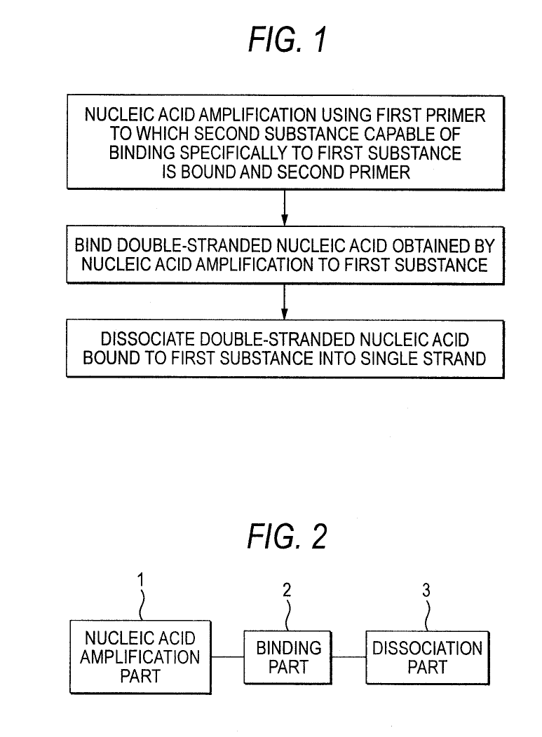

Next, the separation method of single-stranded nucleic acid of the invention will be described with reference to a flowchart of FIG. 1.

1) Nucleic acid amplification is performed using a first primer to which a second substance capable of binding specifically to a first substance is bound and a second primer to which the second substance is not bound.
2) Double-stranded nucleic acid obtained by the nucleic acid amplification is bound to the first substance.
3) The double-stranded nucleic acid bound to the first substance is dissociated into a single strand.

Further, an apparatus for carrying out the separation method of single-stranded nucleic acid of the invention is not particularly limited, but an apparatus characterized by having a nucleic acid amplification part 1 for performing nucleic acid amplification using a first primer to which a second substance capable of binding specifically to a first substance is bound and a second primer to which the second substance is not bound, a binding part 2 for binding double-stranded nucleic acid obtained by the nucleic acid amplification to the first substance, and a dissociation part 3 for dissociating the double-stranded nucleic acid bound to the first substance into a single strand as shown in FIG. 2 is given.

In the separation method and apparatus of single-stranded nucleic acid of the invention, a first substance and a second substance are not particularly limited as long as the substances can bind specifically to each other. Concretely, combinations of avidin-biotin, antigen (peptide, etc.)-antibody, ligand-receptor, gold-SH (thiol group), or combinations of relation of covalent binding of amino group-carboxyl group/succinimide/isothiocyanate/isocyanate/hydrazide/acid anhydride/epoxy/aldehyde/triazine/alkyl halide/imido ester, thiol group-maleimide/disulfide/iodoacetamide/haloacetyl, etc. are given. Among them, the combination of avidin-biotin is both in vivo substances and is harmless and safe and is easy to handle, so that this is preferable. Further, it is preferable that the first substance be avidin and the second substance be biotin among them. This is because avidin has four binding sites of biotin, and thereby four (four molecular) double-stranded nucleic acids to which biotin binds can be bound by one molecular avidin, and efficiency of supplement and recovery of double-stranded nucleic acid in the invention improves.

In the separation method and apparatus of single-stranded nucleic acid of the invention, a technique of nucleic acid amplification is not particularly limited. Concretely, various techniques of PCR, LAMP, ICAN, etc. are given. The general PCR is preferable among them.

In the separation method and apparatus of single-stranded nucleic acid of the invention, a technique for dissociating double-stranded nucleic acid bound to a first substance into a single strand is not particularly limited. Concretely, various techniques of alkali treatment, heating treatment, salt concentration manipulation, etc. are given. The alkali treatment is preferable among them since the treatment is the simplest.

In the separation method and apparatus of single-stranded nucleic acid of the invention, a carrier may be bound to a first substance. A separation efficiency of single-stranded nucleic acid of the invention improves by using the carrier.

The carrier is not particularly limited and concretely, a magnetic material, a bead, a basal plate, a fiber, etc. are given. A magnetic particle is preferable as the carrier. In the case of the magnetic particle, by applying external magnetic field after the magnetic particles are dispersed in liquid in which obtained double-stranded nucleic acid (to which a second substance is bound) or single-stranded nucleic acid (to which a second substance is bound) after the obtained double-stranded nucleic acid is dissociated into a single strand is present, the double-stranded nucleic acid or the single-stranded nucleic acid can be effectively fixed, supplemented, recovered and separated.

Further, when the bead is used as the carrier, fixation, supplement, recovery and separation can be performed by filter filtration or gel filtration.

As described above, the invention can efficiently prepare the single-stranded nucleic acid using the magnetic particles, and also has an advantage capable of purification manipulations, for example, removal of protein in reaction liquid of PCR etc. or removal of unrecovered double-stranded nucleic acid.

Preferable embodiments of the invention will be described below.

### FIRST EMBODIMENT

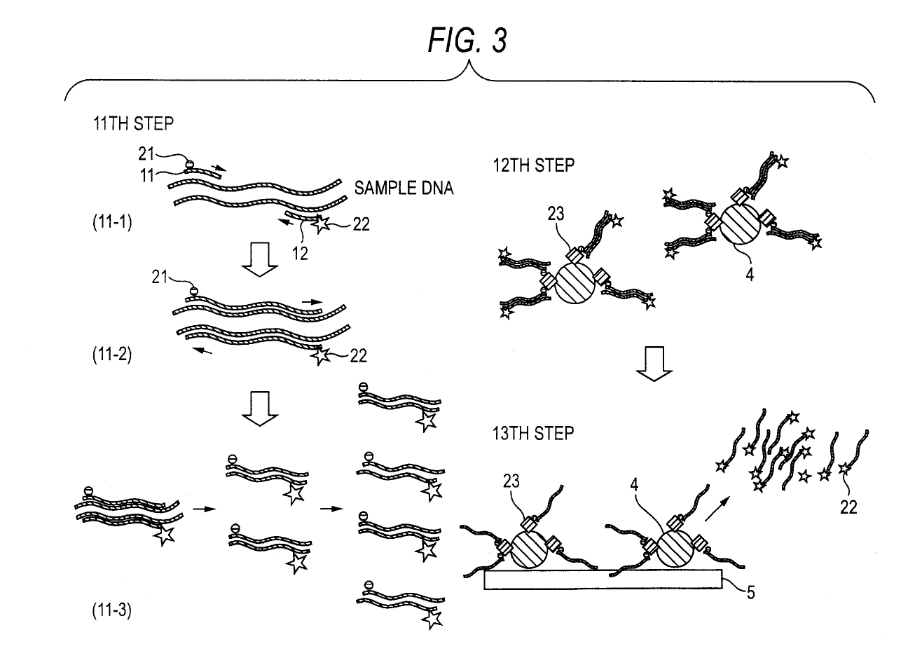

As shown in FIG. 3, PCR amplification of DNA which is nucleic acid is performed using a primer to which biotin 21 binds as a first primer 11 and a primer having a fluorescent substance 22 as a second primer 12 (11th step: 11-1st to 11-3rd steps). Double-stranded DNA obtained by the PCR amplification is bound to avidins 23 bound to a magnetic particle 4 (12th step). The magnetic particles 4 to which the double-stranded DNA is bound are recovered and fixed by a magnet 5, and the double-stranded DNA is dissociated into single strands by alkali treatment (13th step). The single-stranded DNA labeled with the fluorescent substance 22 is present in liquid, and can easily be separated and recovered by recovering the supernatant and can be used as a fluorescence-labeled target in the case of analyzing gene information by hybridization using a DNA microarray or a DNA chip, etc. as it is.

### SECOND EMBODIMENT

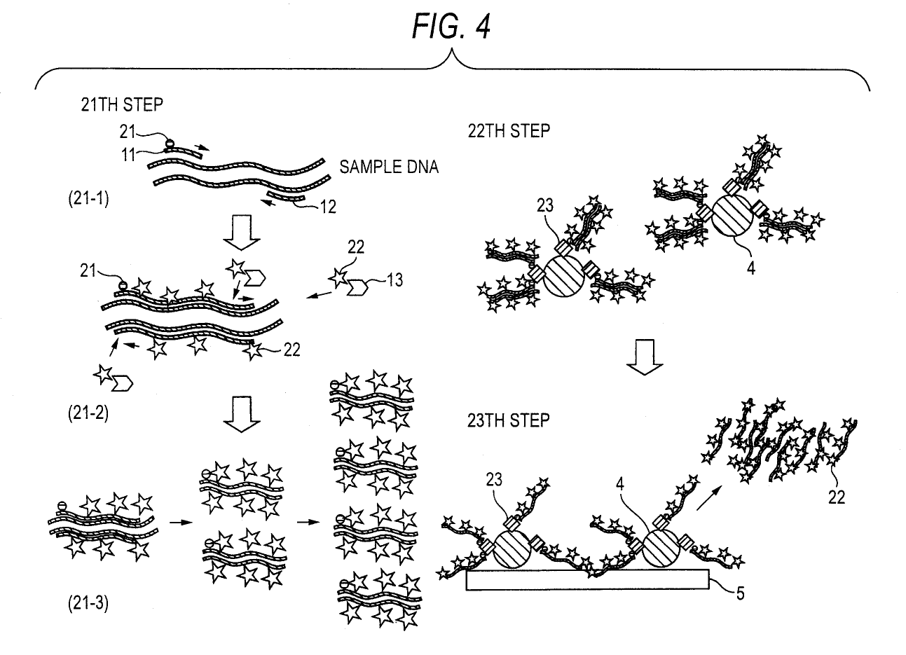

As shown in FIG. 4, PCR amplification of DNA is performed using a primer to which biotin 21 binds as a first primer 11 and a primer without modification as a second primer 12 and dNTP 13 having a fluorescent substance 22 as nucleotide (21st step: 21-1st to 21-3rd steps). Double-stranded DNA obtained by the PCR amplification is bound to avidins 23 bound to a magnetic particle 4 (22nd step). The magnetic particles 4 to which the double-stranded DNA is bound are recovered and fixed by a magnet 5, and the double-stranded DNA is dissociated into single strands by alkali treatment (23rd step). The single-stranded DNA labeled with the fluorescent substance 22 is present in liquid, and can easily be separated and recovered by recovering the supernatant and can be used as a fluorescence-labeled target in the case of analyzing gene information by hybridization using a DNA microarray or a DNA chip, etc. as it is.

In addition, in the first and second embodiments described above, amplification of nucleic acid is performed using the fluorescence-labeled primer or the fluorescence-labeled nucleotide, and fluorescence-labeled single-stranded nucleic acid is obtained and this fluorescence-labeled single-stranded nucleic acid can be used as a target etc. as it is, but the invention does not necessarily obtain the labeled single-stranded nucleic acid using the fluorescence-labeled primer and the fluorescence-labeled nucleotide, etc. at the time of amplification of nucleic acid. Unlabeled single-stranded nucleic acid is obtained using an unlabeled primer or nucleotide at the time of amplification of nucleic acid and this unlabeled single-stranded nucleic acid is used in a target etc. and hybridization is performed and then, a detection reagent or a labeled substance having the so-called intercalation action in which double-stranded nucleic acid is specifically recognized and is intercalated between the double strands can also be used.

Further, as other measurement forms in which a labeled substance is not used at the time of amplification of nucleic acid, there is a form in which a DNA microarray or a DNA chip, etc. are configured to have many electrodes on a basal plate and fix respective different probe nucleic acids to each of the electrodes and connect a current source and detection can be performed by measuring a difference between current amounts in the electrode in which a target is hybridized and the electrode in which a target is not hybridized.

### EXPERIMENT DATA

A PCR product (double-stranded DNA) using a substance obtained by binding biotin (second substance) to only one primer (first primer) is sampled.

The PCR product is mixed with a magnetic particle (carrier) to which streptavidin (first substance) is bound, and the double-stranded DNA is bound to the magnetic particle.

After the magnetic particle is magnetically fixed, supernatant is removed and NaOH is added (treated) to the supernatant and single strands of the double-stranded DNA are formed.

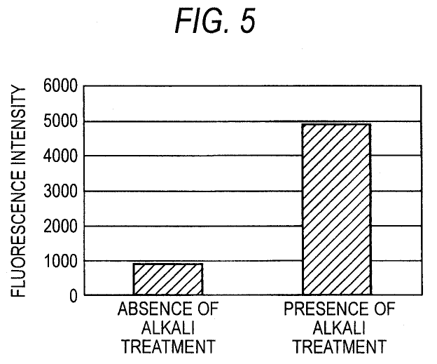

A single-stranded DNA detection reagent (OliGreen: MolecularProbes, Inc.) is added to the supernatant after the addition of NaOH and the fluorescence intensity is measured. The measurement result is shown in FIG. 5.

As shown in FIG. 5, as compared with a sample stained with the single-stranded DNA detection reagent without performing NaOH treatment, a quintuple difference between the sample and a sample with performing NaOH treatment in the fluorescence intensity is obtained. Therefore, it is found that the single-stranded DNA can be separated in the supernatant by alkali treatment.

A separation method of single-stranded nucleic acid of the invention is used in construction of a target in the case of analyzing gene information using a DNA chip or a microarray. In other words, the single-stranded nucleic acid obtained by the method of the invention is used as the target in the case of analyzing gene information using the DNA chip or the microarray, etc.

Further, the separation method of single-stranded nucleic acid of the invention is used in construction of a probe for constructing a microarray for analyzing gene information. In other words, the single-stranded nucleic acid obtained by the method of the invention is used as the probe for constructing the microarray for analyzing gene information.

In the separation method of single-stranded nucleic acid of the invention, the single-stranded nucleic acid of a target etc. used in the case of analyzing gene information using a DNA microarray or a DNA chip, etc. can be prepared and separated without performing centrifugation, so that a cartridge of an instrument for carrying out the method of the invention can be formed.

A form of a cartridge for carrying out the method of the invention is not particularly limited, but it may be a form of performing only the minimum steps ranging to amplification of purified nucleic acid, supplement and recovery and fixation, and single-strand formation, and also may be a form capable of extraction of nucleic acid from blood and body tissue, etc., hybridization of a target to a DNA microarray or a DNA chip, etc., and further detection by a reading apparatus.

1. A separation method of single-stranded nucleic acid, comprising the steps of:
performing nucleic acid amplification using a first primer to which a second substance capable of binding specifically to a first substance is bound and a second primer to which the second substance is not bound, and
binding double-stranded nucleic acid obtained by the nucleic acid amplification to the first substance, and
dissociating the double-stranded nucleic acid bound to the first substance into a single strand.

2. The separation method of single-stranded nucleic acid as claimed in claim 1, wherein
the double-stranded nucleic acid is dissociated into a single strand by alkali treatment.

3. The separation method of single-stranded nucleic acid as claimed in claim 1, wherein
the first substance is avidin, and
the second substance is biotin.

4. The separation method of single-stranded nucleic acid as claimed in claim 1, wherein
the first substance is an antigen, and
the second substance is an antibody.

5. The separation method of single-stranded nucleic acid as claimed in claim 1, wherein
the first substance is gold, and
the second substance is thiol.

6. The separation method of single-stranded nucleic acid as claimed in claim 1, wherein
the first substance is a substance having an amino group, and
the second substance is a substance having a group binding covalently to an amino group.

7. The separation method of single-stranded nucleic acid as claimed in claim 1, wherein
a carrier is bound to the first substance.

8. The separation method of single-stranded nucleic acid as claimed in claim 7, wherein
the carrier is one of a magnetic particle, a bead, a basal plate and a fiber.

9. The separation method of single-stranded nucleic acid as claimed in claim 1, wherein
the second primer has a labeled substance.

10. The separation method of single-stranded nucleic acid as claimed in claim 9, wherein
the labeled substance is a fluorescent substance.

11. The separation method of single-stranded nucleic acid as claimed in claim 1, wherein
nucleotide used in the nucleic acid amplification has a labeled substance.

12. The separation method of single-stranded nucleic acid as claimed in claim 11, wherein
the labeled substance is a fluorescent substance.

13. A separation apparatus of single-stranded nucleic acid comprising:
a nucleic acid amplification part for performing nucleic acid amplification with using a first primer to which a second substance capable of binding specifically to a first substance is bound and a second primer to which the second substance is not bound,
a binding part for binding double-stranded nucleic acid obtained by the nucleic acid amplification to the first substance, and
a dissociation part for dissociating the double-stranded nucleic acid bound to the first substance into a single strand.

14. A microarray having single-stranded nucleic acid obtained by the separation method of single-stranded nucleic acid as claimed in claim 1 as a probe.

15. A microarray wherein single-stranded nucleic acid obtained by the separation method of single-stranded nucleic acid as claimed in claim 1 is hybridized as a target.

16. A DNA chip wherein single-stranded nucleic acid obtained by the separation method of single-stranded nucleic acid as claimed in claim 1 is hybridized as a target.
